(19) United States
(12) Patent Application Publication
(10) Pub. No.: US 2023/0218526 A1
(43) Pub. Date: Jul. 13, 2023

(54) COLD FILTRATION OF OIL-IN-WATER EMULSION ADJUVANTS

(71) Applicant: Seqirus UK Limited, Berkshire (GB)

(72) Inventor: Stephen HUNN, Berkshire (GB)

(21) Appl. No.: 18/001,802

(22) PCT Filed: Jun. 29, 2021
(86) PCT No.: PCT/IB2021/055810

§ 371 Date: Dec. 14, 2022

Related U.S. Application Data
(60) Provisional application No. 63/045,949, filed on Jun. 30, 2020.

(51) Int. Cl. A61K 9/107; A61K 39/39; A61K 47/14

(52) U.S. Cl. CPC A61K 9/1075; A61K 39/39; A61K 47/14; A61K 2039/55566

(57) ABSTRACT

The present disclosure relates to the method of filtering emulsions at cold temperatures. Specifically, cold filtration of emulsion adjuvants for vaccine manufacture is discussed.

### CROSS-REFERENCE

This application claims the benefit of priority of U.S. Provisional Patent Application No. 63/045,949, filed Jun. 30, 2020, the entire contents of which are incorporated herein by reference.

### FIELD

This invention is in the field of manufacturing oil-in-water emulsions for vaccines. The present disclosure relates to the method of filtering oil-in-water emulsions at decreased temperatures. Additionally, filtration of oil-in-water emulsions at decreased temperatures for vaccine manufacture is discussed.

### BACKGROUND

Pharmaceutical or immunological agents that increase the immune response to antigens are important for vaccine manufacture (Rogers et al. (2010) BioPharm International Supplement, Issue 1:1-4). Oil-in-water emulsion that can be utilized as adjuvants are one such example of agents that enhance immune responses (Rogers et al. (2010) BioPharm International Supplement, Issue 1:1-4). The use of these adjuvants in vaccine formulation is advantageous because adjuvants in vaccine formulations enhance, accelerate, and prolong vaccine efficacy (Rogers et al. (2010) BioPharm International Supplement, Issue 1:1-4; Onraedt et al. (2010) BioPharm International Supplement, Issue 8). Adjuvants have also been described as dose-sparing because they elicit quicker and broader responses during pandemic outbreaks (Onraedt et al. (2010) BioPharm International Supplement, Issue 8). Oil-in-water emulsions and liposome adjuvants, for instance, are being pursued by vaccine manufacturers worldwide as a cost-effective mechanism to meet worldwide vaccine demand (Rogers et al. (2010) BioPharm International Supplement, Issue 1:1-4).

Emulsions have previously been described as thermodynamically unstable (Raposo et al. (2013) Pharm Dev Technol 1-13). Potential stabilizers include multi-functional excipients, such as surfactants, co-emulsifiers, polymers, biomolecules, and colloidal particles (Raposo et al. (2013) Pharm Dev Technol 1-13; Tamilvanan et al. (2010) J. Excipients and Food Chem 1(1):11-29).

One such oil-in-water adjuvant is known as ‘MF59’® (WO90/14837; Podda & Del Giudice (2003) Expert Rev Vaccines 2:197-203; Podda (2001) Vaccine 19:2673-2680). MF59® is a submicron oil-in-water emulsion of squalene, polysorbate 80 (also known as Tween 80), and sorbitan trioleate (also known as Span 85). It may also include citrate ions e.g. 10 mM sodium citrate buffer (Vaccine Design: The Subunit and Adjuvant Approach (eds. Powell & Newman) Plenum Press 1995 (ISBN 0-306-44867-X; Vaccine Adjuvants: Preparation Methods and Research Protocols (Volume 42 of Methods in Molecular Medicine series). ISBN: 1-59259-083-7. Ed. O'Hagan; New Generation Vaccines (eds. Levine et al). 3rd edition, 2004. ISBN 0-8247-4071-8). The composition of the emulsion by volume can be about 5% squalene, about 0.5% Tween 80 and about 0.5% Span 85 (Vaccine Design: The Subunit and Adjuvant Approach (eds. Powell & Newman) Plenum Press 1995 (ISBN 0-306-44867-X; Vaccine Adjuvants: Preparation Methods and Research Protocols (Volume 42 of Methods in Molecular Medicine series). ISBN: 1-59259-083-7. Ed. O'Hagan; New Generation Vaccines (eds. Levine et al). 3rd edition, 2004. ISBN 0-8247-4071-8).

MF59® is manufactured on a commercial scale by dispersing Span 85 in the squalene phase and Tween 80 in the aqueous phase, followed by high-speed mixing to form a coarse emulsion (O'Hagan (2007) Expert Rev Vaccines 6(5):699-710). This coarse emulsion is then repeatedly passed through a microfluidizer to produce an emulsion having a uniform oil droplet size (O'Hagan (2007) Expert Rev Vaccines 6(5):699-710). The microfluidized emulsion is then filtered through a 0.22 μm membrane to remove large oil droplets, and the mean droplet size of the resulting emulsion remains unchanged for at least 3 years at 4° C. (New Generation Vaccines (eds. Levine et al). 3rd edition, 2004. ISBN 0-8247-4071-8). The squalene content of the final emulsion is then measured (EP-B-2029170).

Throughput of an oil-in-water emulsion through a membrane in a typical filtration application can be affected by many factors, including membrane structure, viscosity of the adjuvant suspension, adjuvant particle size, adjuvant particle concentration, and resistance of the filter material (Rogers et al. (2010) BioPharm International Supplement, Issue 1:1-4). The filter's overall throughput is determined by flux and capacity (Rogers et al. (2010) BioPharm International Supplement, Issue 1:1-4). Flux is determined by driving forces (e.g. inlet pressure), stream properties (viscosity), and the membrane structure (e.g. pore size, asymmetry) (Rogers et al. (2010) BioPharm International Supplement, Issue 1:1-4). A reduced flux can significantly affect processing time (Rogers et al. (2010) BioPharm International Supplement, Issue 1:1-4). Capacity is driven by membrane structure and by properties of the process stream, such as adjuvant particle load (Rogers et al. (2010) BioPharm International Supplement, Issue 1:1-4). Both asymmetric membranes and increased pressure have previously been associated with enhancing membrane capacity (Rogers et al. (2010) BioPharm International Supplement, Issue 1:1-4).

Regarding viscosity, suspensions are typically less viscous at higher temperatures but, at all temperatures, viscosity is higher than that of water (Rogers et al. (2010) BioPharm International Supplement, Issue 1:1-4). Flux of more viscous solutions is higher than that of aqueous solutions (Rogers et al. (2010) BioPharm International Supplement, Issue 1:1-4).

Membrane plugging is another factor meriting considering during filtration of emulsions. Flow will typically decline quickly after filtration commences because of particle plugging of membranes and because of the particulate character of adjuvants (Rogers et al. (2010) BioPharm International Supplement, Issue 1:1-4). Thus, membrane pore blockage is an important factor in filter capacity and is a primary mechanism of flow decay (Rogers et al. (2010) BioPharm International Supplement, Issue 1:1-4). Flow of smaller particles has previously been linked to increased membrane capacity (Rogers et al. (2010) BioPharm International Supplement, Issue 1:1-4).

Retention of extraneous contaminants such as bacteria is another key consideration during membrane filtration of emulsions. Multiple factors have been associated with influencing bacterial retention, including the interaction between the adjuvant, bacteria, and the membrane; membrane plugging; adjuvant surface tension; membrane properties; temperature; and operating pressure (Onraedt et al. (2010) BioPharm International Supplement, Issue 8). The coating of bacteria with emulsion has been linked to less robust retention, as have membrane pore blockage and low adjuvant surface tension (Onraedt et al. (2010) BioPharm International Supplement, Issue 8). Raising temperature has been associated with increased retention (Onraedt et al. (2010) BioPharm International Supplement, Issue 8).

One of the mechanisms disclosed in the prior art employed to circumvent the many issues associated with membrane filtration of emulsions involves heating the emulsions prior to filtration (Tamilvanan et al. (2010) J. Excipients and Food Chem 1(1):11-29). Increased emulsion temperature has been associated with enhanced filtration but can significantly damage the integrity of emulsions as well as their subsequent performance in vaccines.

Preparation of oil-in-water emulsions, such as MF59®, typically involves multiple levels of filtration such as, bioburden reduction filtration, sterile filtration, particles size filtrations, and so forth. In a manufacturing context, these filtration steps utilize a large amount of filter membranes. In view of this, improvements in filtration methods and systems are needed.

### SUMMARY

The disclosure provides for emulsion adjuvants subjected to membrane filtration at cold temperatures.

The disclosure also provides for methods of filtering emulsion adjuvants at cold temperatures.

### DESCRIPTION OF DRAWINGS

FIG. 1 depicts throughput at 5° C., 30° C., and 40° C. temperatures for an SHF membrane.

FIG. 2 depicts throughput at 5° C., 30° C., and 40° C. for an SHC membrane.

FIG. 3 depicts throughput at 5° C. and 40° C. for an ECV membrane.

### DETAILED DESCRIPTION

Many modifications and other embodiments of the disclosures set forth herein will come to mind to one skilled in the art to which these disclosures pertain having the benefit of the teachings presented in the foregoing descriptions and the associated drawings. Therefore, it is to be understood that the disclosures are not to be limited to the specific embodiments disclosed and that modifications and other embodiments are intended to be included within the scope of the appended claims. Although specific terms are employed herein, they are used in a generic and descriptive sense only and not for purposes of limitation.

As used herein, the singular forms “a,” “an,” and “the” are intended to include the plural forms as well, unless the context clearly indicates otherwise. Furthermore, to the extent that the terms “including,” “includes,” “having,” “has,” “with,” or variants thereof are used in either the detailed description and/or the claims, such terms are intended to be inclusive in the same manner as the term “comprising.”

The terms “comprise,” “have” and “include” are open-ended linking verbs. Any forms or tenses of one or more of these verbs, such as “comprises,” “comprising,” “has,” “having,” “includes” and “including,” are also open-ended. For example, any method that “comprises,” “has” or “includes” one or more steps is not limited to possessing only those one or more steps and can also cover other unlisted steps. Similarly, any composition that “comprises,” “has” or “includes” one or more features is not limited to possessing only those one or more features and can cover other unlisted features. The use of any and all examples, or exemplary language (e.g., “such as”) provided with respect to certain embodiments herein is intended merely to better illuminate the present disclosure and does not pose a limitation on the scope of the present disclosure otherwise claimed.

### Oil-In-Water Emulsion Adjuvant

Methods of the invention are used for the manufacture of oil-in-water emulsions. These emulsions include three core ingredients: an oil; an aqueous component; and a surfactant.

Oil-in-water emulsions have been found to be suitable for use as adjuvants in influenza virus vaccines. Various such emulsions are known, and they typically include at least one oil and at least one surfactant, with the oil(s) and surfactant(s) being biodegradable (metabolizable) and biocompatible. The oil droplets in the emulsion are generally less than 5 μm in diameter, and may even have a sub-micron diameter, with these small sizes being achieved with a microfluidizer to provide stable emulsions. Droplets with a size less than 220 nm are preferred as they can be subjected to filter sterilization.

The oils can be from an animal (such as fish) or vegetable source. Because the emulsions are intended for pharmaceutical use then the oil will typically be biodegradable (metabolisable) and biocompatible. Sources for vegetable oils include nuts, seeds and grains. Peanut oil, soybean oil, coconut oil, and olive oil, the most commonly available, exemplify the nut oils. Jojoba oil can be used, e.g., obtained from the jojoba bean. Seed oils include safflower oil, cottonseed oil, sunflower seed oil, sesame seed oil and the like. In the grain group, corn oil is the most readily available, but the oil of other cereal grains such as wheat, oats, rye, rice, teff, triticale and the like may also be used. 6-10 carbon fatty acid esters of glycerol and 1,2-propanediol, while not occurring naturally in seed oils, may be prepared by hydrolysis, separation and esterification of the appropriate materials starting from the nut and seed oils. Fats and oils from mammalian milk are metabolizable and may therefore be used in the practice of this invention. The procedures for separation, purification, saponification and other means necessary for obtaining pure oils from animal sources are well known in the art. A number of branched chain oils are synthesized biochemically in 5-carbon isoprene units and are generally referred to as terpenoids. Shark liver oil contains a branched, unsaturated terpenoid known as squalene, 2,6,10,15,19,23-hexamethyl-2,6,10,14,18,22-tetracosahexaene. Squalane, the saturated analog to squalene, is another example of oil. The oil of the present invention may comprise a mixture (or combination) of oils e.g. comprising squalene and at least one further oil. Fish oils, including squalene and squalane, are readily available from commercial sources or may be obtained by methods known in the art.

Other useful oils are the tocopherols, particularly in combination with squalene. Where the oil phase of an emulsion includes a tocopherol, any of the α, β, γ, δ, ε or ζ, tocopherols can be used, but α-tocopherols are preferred. D-α-tocopherol and DL-α-tocopherol can both be used. A preferred α-tocopherol is DL-α-tocopherol. The tocopherol can take several forms e.g. different salts and/or isomers. Salts include organic salts, such as succinate, acetate, nicotinate, etc. If a salt of this tocopherol is to be used, the preferred salt is the succinate. An oil combination comprising squalene and a tocopherol (e.g. DL-α-tocopherol) can be used.

The aqueous component can be plain water (e.g. w.f.i.) or can include further components e.g. solutes. For instance, it may include salts to form a buffer e.g. citrate or phosphate salts, such as sodium salts. Typical buffers include: a phosphate buffer; a Tris buffer; a borate buffer; a succinate buffer; a histidine buffer; or a citrate buffer. Buffers will typically be included in the 5-20 mM range.

The surfactant is preferably biodegradable (metabolisable) and biocompatible. Surfactants can be classified by their ‘HLB’ (hydrophile/lipophile balance), where a HLB in the range 1-10 generally means that the surfactant is more soluble in oil than in water, and a HLB in the range 10-20 are more soluble in water than in oil. Emulsions preferably comprise at least one surfactant that has a HLB of at least 10 e.g. at least 15, or preferably at least 16.

The invention can be used with surfactants including, but not limited to: the polyoxyethylene sorbitan esters surfactants (commonly referred to as the Tweens), especially polysorbate 20 and polysorbate 80; copolymers of ethylene oxide (EO), propylene oxide (PO), and/or butylene oxide (BO), sold under the DOWFAX™ tradename, such as linear EO/PO block copolymers; octoxynols, which can vary in the number of repeating ethoxy (oxy-1,2-ethanediyl) groups, with octoxynol-9 (Triton X-100, or t-octylphenoxypolyethoxyethanol) being of particular interest; (octylphenoxy)polyethoxyethanol (IGEPAL CA-630/NP-40); phospholipids such as phosphatidylcholine (lecithin); polyoxyethylene fatty ethers derived from lauryl, cetyl, stearyl and oleyl alcohols (known as Brij surfactants), such as triethyleneglycol monolauryl ether (Brij 30); polyoxyethylene-9-lauryl ether; and sorbitan esters (commonly known as the SPANs), such as sorbitan trioleate (Span 85) and sorbitan monolaurate. Preferred surfactants for including in the emulsion are polysorbate 80 (Tween 80; polyoxyethylene sorbitan monooleate), Span 85 (sorbitan trioleate), lecithin and Triton X-100.

Mixtures of surfactants can be included in the emulsion e.g. Tween 80/Span 85 mixtures, or Tween 80/Triton-X 100 mixtures. A combination of a polyoxyethylene sorbitan ester such as polyoxyethylene sorbitan monooleate (Tween 80) and an octoxynol such as t-octylphenoxy-polyethoxyethanol (Triton X-100) is also suitable. Another useful combination comprises laureth 9 plus a polyoxyethylene sorbitan ester and/or an octoxynol. Useful mixtures can comprise a surfactant with a HLB value in the range of 10-20 (e.g. Tween 80, with a HLB of 15.0) and a surfactant with a HLB value in the range of 1-10 (e.g. Span 85, with a HLB of 1.8).

Suitable amounts of surfactants (% by weight) are: polyoxyethylene sorbitan esters (such as Tween 80) 0.01 to 1%, in particular about 0.1%; octyl- or nonylphenoxy polyoxyethanols (such as Triton X-100, or other detergents in the Triton series) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%.

Whatever the choice of oil(s) and surfactant(s), the surfactant(s) is/are included in excess of the amount required for emulsification, such that free surfactant remains in the aqueous phase. Free surfactant in the final emulsion can be detected by various assays. For instance, a sucrose gradient centrifugation method can be used to separate emulsion droplets from the aqueous phase, and the aqueous phase can then be analyzed. Centrifugation can be used to separate the two phases, with the oil droplets coalescing and rising to the surface, after which the surfactant content of the aqueous phase can be determined, e.g., using HPLC or any other suitable analytical technique.

Specific oil-in-water emulsion adjuvants according to this disclosure include, but are not limited to, the following:
A submicron emulsion of squalene, Tween 80, and Span 85. The composition of the emulsion by volume can be about 5% squalene, about 0.5% polysorbate 80 and about 0.5% Span 85. In weight terms, these ratios become 4.3% squalene, 0.5% polysorbate 80 and 0.48% Span 85. This adjuvant is known as “MF59”®. The MF59® emulsion advantageously includes citrate ions, e.g., 10 mM sodium citrate buffer. In some embodiments, the oil-in-water elusion adjuvant is a squalene-in-water emulsion adjuvant having 9.75 mg squalene.
An emulsion of squalene, a tocopherol, and Tween 80. The emulsion may include phosphate buffered saline. It may also include Span 85 (e.g., at 1%) and/or lecithin. These emulsions may have from 2 to 10% squalene, from 2 to 10% tocopherol and from 0.3 to 3% Tween 80, and the weight ratio of squalene:tocopherol is preferably ≤1 as this provides a more stable emulsion. Squalene and Tween 80 may be present volume ratio of about 5:2. One such emulsion can be made by dissolving Tween 80 in PBS to give a 2% solution, then mixing 90 mL of this solution with a mixture of (5 g of DL-α-tocopherol and 5 mL squalene), then microfluidizing the mixture. The resulting emulsion may have submicron oil droplets, e.g. with an average diameter of between 100 and 250 nm, preferably about 180 nm.
An emulsion of squalene, a tocopherol, and a Triton detergent (e.g., Triton X-100). The emulsion may also include a 3d-MPL. The emulsion may contain a phosphate buffer.
An emulsion comprising a polysorbate (e.g., polysorbate 80), a Triton detergent (e.g., Triton X-100) and a tocopherol (e.g., an α-tocopherol succinate). The emulsion may include these three components at a mass ratio of about 75:11:10 (e.g., 750 μg/mL polysorbate 80, 110 μg/mL Triton X-100 and 100 μg/mL α-tocopherol succinate), and these concentrations should include any contribution of these components from antigens. The emulsion may also include squalene. The emulsion may also include a 3d-MPL. The aqueous phase may contain a phosphate buffer.
An emulsion of squalane, polysorbate 80 and poloxamer 401 (“Pluronic™ L121”). The emulsion can be formulated in phosphate buffered saline, pH 7.4. This emulsion is a useful delivery vehicle for muramyl dipeptides, and has been used with threonyl-MDP in the “SAF-1” adjuvant (0.05-1% Thr-MDP, 5% squalane, 2.5% Pluronic L121 and 0.2% polysorbate 80). It can also be used without the Thr-MDP, as in the “AF” adjuvant (5% squalane, 1.25% Pluronic L121 and 0.2% polysorbate 80).
An emulsion having from 0.5-50% of an oil, 0.1-10% of a phospholipid, and 0.05-5% of a non-ionic surfactant. Preferred phospholipid components are phosphatidylcholine, phosphatidylethanolamine, phosphatidylserine, phosphatidylinositol, phosphatidylglycerol, phosphatidic acid, sphingomyelin and cardiolipin. Submicron droplet sizes are advantageous.
A submicron oil-in-water emulsion of a non-metabolizable oil (such as light mineral oil) and at least one surfactant (such as lecithin, Tween 80 or Span 80). Additives may be included, such as QuilA saponin, cholesterol, a saponin-lipophile conjugate (such as GPI-0100, produced by addition of aliphatic amine to desacylsaponin via the carboxyl group of glucuronic acid), dimethyidioctadecylammonium bromide and/or N,N-dioctadecyl-N,N-bis (2-hydroxyethyl)propanediamine.
An emulsion in which a saponin (e.g., QuilA or QS21) and a sterol (e.g., a cholesterol) are associated as helical micelles.

### Formation of the Emulsion

Emulsion components may be mixed to form an emulsion.

Oil droplets in the emulsion may have an average size of 5000 nm or less e.g. 4000 nm or less, 3000 nm or less, 2000 nm or less, 1200 nm or less, 1000 nm or less, e.g. an average size between 800 and 1200 nm or between 300 nm and 800 nm.

The number of oil droplets in the emulsion with a size>1.2 μm may be 5×10¹¹/ml or less, e.g. 5×10¹⁰/ml or less or 5×10⁹/ml or less.

The average oil droplet size of the emulsion can be achieved by mixing the first emulsion's components in a homogenizer. Homogenizers can operate in a vertical and/or horizontal manner. For convenience in a commercial setting, in-line homogenizers are preferred.

For commercial-scale manufacture the homogenizer should ideally have a flow rate of at least 300 L/hr e.g. ≥400 L/hr, ≥500 L/hr, ≥600 L/hr, ≥700 L/hr, ≥800 L/hr, ≥900 L/hr, ≥1000 L/hr, ≥2000 L/hr, ≥5000 L/hr, or even ≥10000 L/hr. Suitable high-capacity homogenizers are commercially available.

A preferred homogenizer provides a shear rate of between 3×10⁵ and 1×10⁶ s⁻¹, e.g. between 3×10⁵ and 7×10⁵ s⁻¹, between 4×10⁵ and 6×10⁵ s⁻¹, e.g. about 5×10⁵ s⁻¹.

In some embodiments the emulsion components may be homogenized multiple times (e.g. 2, 3, 4, 5, 6, 7, 8, 9, 10, 20, 30, 40, 50 or more times). To avoid the need for a long string of containers and homogenizers the emulsion components can be circulated. In particular, the emulsion may be formed by circulating the first emulsion components through a homogenizer a plurality of times (e.g. 2, 3, 4, 5, 6, 7, 8, 9, 10, 20, 30, 40, 50, 100 etc times). However, too many cycles may be undesirable as it can produce re-coalescence (Jafari et al. (2008) Food Hydrocolloids 22:1191-1202). Thus, the size of oil droplets may be monitored if homogenizer circulation is used to check that a desired droplet size is reached and/or that re-coalescence is not occurring.

Circulation through the homogenizer is advantageous because it can reduce the average size of the oil droplets in the emulsion. Circulation is also advantageous because it can reduce the number of oil droplets having a size>1.2 μm in the first emulsion. These reductions in average droplet size and number of droplets>1.2 μm in the first emulsion can provide advantages in downstream process(es). In particular, circulation of the emulsion components through the homogenizer can lead to an improved microfluidization process, which, itself, can provide improved filtration performance. Improved filtration performance may lead to less content losses during filtration, e.g. losses of squalene, Tween 80 and Span 85 when the oil-in-water emulsion is MF59®.

Methods of the invention may be used at large scale. Thus, a method may involve preparing a first emulsion whose volume is greater than 1 liter e.g. ≥5 liters, ≥10 liters, ≥20 liters, ≥50 liters, ≥100 liters, ≥250 liters, etc.

### Microfluidization

After its formation the emulsion may be microfluidized to reduce its average oil droplet size and/or to reduce the number of oil droplets having a size of >1.2 μm.

Microfluidization instruments reduce average oil droplet size by propelling streams of input components through geometrically fixed channels at high pressure and high velocity. The pressure at the entrance to the interaction chamber (also called the “first pressure”) may be substantially constant (i.e. ±15%; e.g. ±10%, ±5%, ±2%) for at least 85% of the time during which components are fed into the microfluidizer, e.g. at least 87%, at least 90%, at least 95%, at least 99% or 100% of the time during which the emulsion is fed into the microfluidizer.

A microfluidization apparatus typically comprises at least one intensifier pump (preferably two pumps, which may be synchronous) and an interaction chamber. The intensifier pump, which is ideally electric-hydraulic driven, provides high pressure (i.e. the first pressure) to force an emulsion into and through the interaction chamber. The synchronous nature of the intensifier pumps may be used to provide the substantially constant pressure of the emulsion discussed above, which means that the emulsion droplets are all exposed to substantially the same level of shear forces during microfluidization.

The reduction in the average oil droplet size and in the number of oil droplets having a size>1.2 μm in the emulsion may provide improved filtration performance. Improved filtration performance may lead to less content losses during filtration, e.g. losses of squalene, Tween 80 and Span 85 when the emulsion is MF59®.

A preferred microfluidization apparatus operates at a pressure between 170 bar and 2750 bar (approximately 2500 psi to 40000 psi) e.g. at about 345 bar, about 690 bar, about 1380 bar, about 2070 bar, etc.

A preferred microfluidization apparatus has an interaction chamber that provides a shear rate in excess of 1×10⁶ s⁻¹ e.g. ≥2.5×10⁶ s⁻¹, ≥5×10⁶ s⁻¹, ≥10⁷ s⁻¹, etc.

A microfluidization apparatus can include multiple interaction chambers that are used in parallel e.g. 2, 3, 4, 5 or more, but it is more useful to include a single interaction chamber.

The result of microfluidization can be an oil-in-water emulsion in which the average size of the oil droplets is 500 nm or less. This average size is particularly useful as it facilitates filter sterilization of the emulsion. Emulsions in which at least 80% by number of the oil droplets have an average size of 500 nm or less, e.g. 400 nm or less, 300 nm of less, 200 nm or less or 165 nm or less, are particularly useful. Furthermore, the number of oil droplets in the emulsion having a size>1.2 μm is 5×10¹⁰/ml or less, e.g. 5×10⁹/ml or less, 5×10⁸/ml or less or 2×10⁸/ml or less.

The emulsion containers in the microfluidization apparatus may be held under an inert gas, e.g. up to 0.5 bar of nitrogen. This prevents the emulsion components oxidizing, which is particularly advantageous if one of the emulsion components is squalene. This leads to an increase in the stability of the emulsion.

Methods of the invention may be used at large scale. Thus, a method may involve microfluidizing a volume greater than 1 liter e.g. ≥5 liters, ≥10 liters, ≥20 liters, ≥50 liters, ≥100 liters, ≥250 liters, etc.

### Filtration

After microfluidization, the emulsion is filtered. Filtration removes any large oil droplets that have survived the homogenization and microfluidization procedures. Although small in terms of overall number, these oil droplets can be large in terms of volume and can act as nucleation sites for aggregation, leading to emulsion degradation during storage. Moreover, filtration can achieve filter sterilization.

Filtration of oil-in-water emulsions in a manufacturing process can include one or multiple levels and/or types of filtration steps. Some of these can include filtering to reduce bioburden, sterile filtration, particle size filtration, and so forth. Thus, in various embodiments, the present disclosure describes methods for improving filtration of emulsions, preferably oil-in-water emulsions. In one or more preferred aspects, the type of filtrations embodied by the present disclosure include, but are not limited to, bioburden reduction filtering, sterile filtering, and particle size filtering.

The particular filtration membrane suitable for filtering depends on the fluid characteristics of the emulsion and the degree of filtration required. A filter's characteristics can affect its suitability for filtration of the microfluidized emulsion. For example, a filter's pore size and surface characteristics can be important, particularly when filtering a squalene-based emulsion.

The pore size of membranes used with the invention should permit passage of the desired droplets while retaining the unwanted droplets. For example, it should retain droplets that have a size of ≥1 μm while permitting passage of droplets<200 nm. A 0.2 μm or 0.22 μm filter is ideal and can also achieve filtering.

The emulsion may be prefiltered e.g. through a 0.45 μm filter. The prefiltration and filtration can be achieved in one step by the use of known double-layer filters that include a first membrane layer with larger pores and a second membrane layer with smaller pores. Double-layer filters are particularly useful with the invention. The first layer ideally has a pore size>0.3 μm, such as between 0.3-2 μm or between 0.3-1 μm, or between 0.4-0.8 μm, or between 0.5-0.7 μm. A pore size of ≤0.75 μm in the first layer is preferred. Thus, the first layer may have a pore size of 0.6 μm or 0.45 μm, for example. The second layer ideally has a pore size which is less than 75% of (and ideally less than half of) the first layer's pore size, such as between 25-70% or between 25-49% of the first layer's pore size e.g. between 30-45%, such as ⅓ or 4/9, of the first layer's pore size. Thus, the second layer may have a pore size<0.3 μm, such as between 0.15-0.28 μm or between 0.18-0.24 μm e.g. a 0.2 μm or 0.22 μm pore size second layer. In one example, the first membrane layer with larger pores provides a 0.45 μm filter, while the second membrane layer with smaller pores provides a 0.22 μm filter.

The filtration membrane and/or the prefiltration membrane may be asymmetric. An asymmetric membrane is one in which the pore size varies from one side of the membrane to the other e.g. in which the pore size is larger at the entrance face than at the exit face. One side of the asymmetric membrane may be referred to as the “coarse pored surface”, while the other side of the asymmetric membrane may be referred to as the “fine pored surface”. In a double-layer filter, one or (ideally) both layers may be asymmetric.

The filtration membrane may be porous or homogeneous. A homogeneous membrane is usually a dense film ranging from 10 to 200 μm. A porous membrane has a porous structure. In one embodiment, the filtration membrane is porous. In a double-layer filter, both layers may be porous, both layers may be homogenous, or there may be one porous and one homogenous layer. A preferred double-layer filter is one in which both layers are porous.

In one embodiment, the emulsion is pre-filtered through an asymmetric, hydrophilic porous membrane and then filtered through another asymmetric hydrophilic porous membrane having smaller pores than the prefiltration membrane. This can use a double-layer filter.

The filter membrane(s) may be autoclaved prior to use to ensure sterility.

Filtration membranes are typically made of polymeric support materials such as PTFE (poly-tetra-fluoro-ethylene), PES (polyethersulfone), PVP (polyvinyl pyrrolidone), PVDF (polyvinylidene fluoride), nylons (polyamides), PP (polypropylene), celluloses (including cellulose esters), PEEK (polyetheretherketone), nitrocellulose, etc. These have varying characteristics, with some supports being intrinsically hydrophobic (e.g. PTFE) and others being intrinsically hydrophilic (e.g. cellulose acetates). However, these intrinsic characteristics can be modified by treating the membrane surface. For instance, it is known to prepare hydrophilized or hydrophobized membranes by treating them with other materials (such as other polymers, graphite, silicone, etc.) to coat the membrane surface (WO90/04609). In a double-layer filter the two membranes can be made of different materials or (ideally) of the same material.

An ideal filter for use with the invention has a hydrophilic surface rather than hydrophobic (polysulfone) surface (Baudner et al. (2009) Pharm Res. 26(6):1477-85; Dupuis et al. (1999) Vaccine 18:434-9; Dupuis et al. (2001) Eur J Immunol 31:2910-8; Burke et al. (1994) J Infect Dis 170:1110-9). Filters with hydrophilic surfaces can be formed from hydrophilic materials, or by hydrophilization of hydrophobic materials, and a preferred filter for use with the invention is a hydrophilic polyethersulfone membrane. Several different methods are known to transform hydrophobic PES membranes into hydrophilic PES membranes. This if often achieved by coating the membrane with a hydrophilic polymer. To provide permanent attachment of the hydrophilic polymer to the PES a hydrophilic coating layer is usually subjected either to a cross-linking reaction or to grafting. A process for modifying the surface properties of a hydrophobic polymer having functionalizable chain ends, comprising contacting the polymer with a solution of a linker moiety to form a covalent link, and then contacting the reacted hydrophobic polymer with a solution of a modifying agent (WO90/04609). A method of PES membrane hydrophilization by direct membrane coating, involving pre-wetting with alcohol, and then soaking in an aqueous solution containing a hydrophilic monomer, a polyfunctional monomer (cross-linker) and a polymerization initiator is additionally used (U.S. Pat. No. 4,618,533). The monomer and cross-linker are then polymerized using thermal- or UV-initiated polymerization to form a coating of cross-linked hydrophilic polymer on the membrane surface (U.S. Pat. No. 4,618,533). Similar methods include coating a PES membrane by soaking it in an aqueous solution of hydrophilic polymer (polyalkylene oxide) and at least one polyfunctional monomer (cross-linker), and then polymerizing a monomer to provide a non-extractable hydrophilic coating (U.S. Pat. Nos. 6,193,077; 6,495,050). The PES membrane can then be hydrophilized by a grafting reaction in which a PES membrane is submitted to low-temperature helium plasma treatment followed by grafting of hydrophilic monomer N-vinyl-2-pyrrolidone (NVP) onto the membrane surface (Chen et al. (1999) Journal of Applied Polymer Science, 72:1699-1711).

In methods that do not rely on coating, PES can be dissolved in a solvent, blended with a soluble hydrophilic additive, and then the blended solution used for casting a hydrophilic membrane e.g. by precipitation or by initiating co-polymerization (U.S. Pat. Nos. 4,943,374; 6,071,406; 4,705,753; 5,178,765; 6,495,043; 6,039,872; 5,277,812). For example, a method of preparing a hydrophilic charge-modified membrane that has low membrane extractables and allows fast recovery of ultrapure water resistivity, having a cross-linked inter-penetrating polymer network structure formed making a polymer solution of a blend of PES, PVP, polyethyleneimine, and aliphatic diglycidyl ether, forming a thin film of the solution, and precipitating the film as a membrane can be employed (U.S. Pat. Nos. 5,277,812; 5,531,893).

Hybrid approaches can be used, in which hydrophilic additives are present during membrane formation and are also added later as a coating (U.S. Pat. No. 4,964,990).

Hydrophilization of PES membrane can also be achieved by treatment with low temperature plasmas, including hydrophilic modification of PES membrane by treatment with low temperature CO₂-plasma (Wavhal & Fisher (2002) Journal of Polymer Science Part B: Polymer Physics 40:2473-88).

Hydrophilization of PES membrane can also be achieved by oxidation (WO2006/044463). This method involves pre-wetting a hydrophobic PES membrane in a liquid having a low surface tension, exposing the wet PES membrane to an aqueous solution of oxidizer, and then heating (WO2006/044463).

Phase inversion can also be used (Espinoza-Gomez et al. (2003) Revista de la Sociedad Quimica de Mexico 47:53-57).

An ideal hydrophilic PES membrane can be obtained by treatment of PES (hydrophobic) with PVP (hydrophilic). Treatment with PEG (hydrophilic) instead of PVP has been found to give a hydrophilized PES membrane that is easily fouled (particularly when using a squalene-containing emulsion) and also disadvantageously releases formaldehyde during autoclaving.

A preferred double-layer filter has a first hydrophilic PES membrane and a second hydrophilic PES membrane.

Known hydrophilic membranes include Bioassure (from Cuno); EverLUX™ polyethersulfone; STyLUX™ polyethersulfone (both from Meissner); Millex GV, Millex HP, Millipak 60, Millipak 200 and Durapore CVGL01TP3 membranes (from Millipore); Fluorodyne™ EX EDF Membrane, Supor™ EAV; Supor™ EBV, Supor™ ECV, Supor™ EKV (all from Pall); Sartopore™ (from Sartorius); Sterlitech's hydrophilic PES membrane; and Wolftechnik's WFPES PES membrane.

During filtration, the emulsion may be maintained at a temperature of 40° C. or less, e.g. 30° C. or less, e.g. 20° C. or less, e.g. 10° C. or less, e.g. 2-8° C. or less, e.g. 5° C. or less to facilitate successful sterile filtration. Some emulsions may not pass through a sterile filter when they are at a temperature of greater than 40° C.

It is advantageous to carry out the filtration step within 24 hours, e.g. within 18 hours, within 12 hours, within 6 hours, within 2 hours, within 30 minutes, of producing the second emulsion because after this time it may not be possible to pass the second emulsion through the sterile filter without clogging the filter (Lidgate et al (1992) Pharmaceutical Research 9(7):860-863).

Methods of the invention may be used at large scale. Thus, a method may involve filtering a volume greater than 1 liter e.g. ≥5 liters, ≥10 liters, ≥20 liters, ≥50 liters, ≥100 liters, ≥250 liters, etc.

In one or more aspects, as described herein, membranes suitable for reduced bioburden can be used in the methods described herein. These membranes can include, but are not limited to: Millipore Milliguard, Pall Supor EAV, Pall Fluorodyne II DBL, Sartorius Sartoguard, etc.

In additional aspects, as described herein, membranes suitable for sterile filtration can be used in the methods described herein. These membranes can include, but are not limited to: Millipore Durapore, Millipore Express SHC, Millipore Express SHF, Pall Supor EBV, Pall Supor ECV, Pall Supor EKV, Pall Emflon II, Pall Fluorodyne II, Pall Fluorodyne EDF, Sartorius Sartopore 2, Sartorius Sartopore 2 XLG, Sartorius Sartopore Platinum, etc.

In further aspects, as described herein, membranes suitable for particle size filtration can be used in the methods described herein. These membranes can include, but are not limited to: Millipore Milliguard, Pall Supor EAV, Pall Fluorodyne II DBL, Pall HDC, Pall Posidyne, Pall PreFlow, Sartorius Sartoguard, Sartorius Sartoclear, etc.

### The Final Emulsion

The result of microfluidization and filtration is an oil-in-water emulsion in which the average size of the oil droplets may be less than 220 nm, e.g. 155±20 nm, 155±10 nm or 155±5 nm, and in which the number of oil droplets having a size>1.2 μm may be 5×10⁸/ml or less, e.g. 5×10⁷/ml or less, 5×10⁶/ml or less, 2×10⁶/ml or less or 5×10⁵/ml or less.

The average oil droplet size of emulsions described herein is generally not less than 50 nm.

Methods of the invention may be used at large scale. Thus, a method may involve preparing a final emulsion with a volume greater than 1 liter e.g. ≥5 liters, ≥10 liters, ≥20 liters, ≥50 liters, ≥100 liters, ≥250 liters, etc.

Once the oil-in-water emulsion has been formed, it may be transferred into sterile glass bottles. The glass bottles may be 5 L, 8 L, or 10 L in size. Alternatively, the oil-in-water may be transferred into a sterile flexible bag (flex bag). The flex bag may be 50 L, 100 L or 250 L in size, etc. In addition, the flex bag may be fitted with one or more sterile connectors to connect the flex bag to the system. The use of a flex bag with a sterile connector is advantageous compared to glass bottles because the flex bag is larger than the glass bottles meaning that it may not be necessary to change the flex bag to store all the emulsion manufactured in a single batch. This can provide a sterile closed system for the manufacture of the emulsion which may reduce the chance of impurities being present in the final emulsion. This can be particularly important if the final emulsion is used for pharmaceutical purposes, e.g. if the final emulsion is the MF59® adjuvant.

Preferred amounts of oil (% by volume) in the final emulsion are between 2-20% e.g. about 10%. A squalene content of about 5% or about 10% is particularly useful. A squalene content (w/v) of between 30-50 mg/ml is useful e.g. between 35-45 mg/ml, 36-42 mg/ml, 38-40 mg/ml, etc.

Preferred amounts of surfactants (% by weight) in the final emulsion are: polyoxyethylene sorbitan esters (such as Tween 80) 0.02 to 2%, in particular about 0.5% or about 1%; sorbitan esters (such as Span 85) 0.02 to 2%, in particular about 0.5% or about 1%; octyl- or nonylphenoxy polyoxyethanols (such as Triton X-100) 0.001 to 0.1%, in particular 0.005 to 0.02%; polyoxyethylene ethers (such as laureth 9) 0.1 to 20%, preferably 0.1 to 10% and in particular 0.1 to 1% or about 0.5%. A polysorbate 80 content (w/v) of between 4-6 mg/ml is useful e.g. between 4.1-5.3 mg/ml. A sorbitan trioleate content (w/v) of between 4-6 mg/ml is useful e.g. between 4.1-5.3 mg/ml.

The process is particularly useful for preparing any of the following oil-in-water emulsions:
An emulsion comprising squalene, polysorbate 80 (Tween 80), and sorbitan trioleate (Span 85). The composition of the emulsion by volume can be about 5% squalene, about 0.5% polysorbate 80 and about 0.5% sorbitan trioleate. In weight terms, these amounts become 4.3% squalene, 0.5% polysorbate 80 and 0.48% sorbitan trioleate. This adjuvant is known as ‘MF59’ ®. The MF59® emulsion advantageously includes citrate ions e.g. 10 mM sodium citrate buffer.
Emulsions comprising squalene, an α-tocopherol (ideally DL-α-tocopherol), and polysorbate 80. These emulsions may have (by weight) from 2 to 10% squalene, from 2 to 10% α-tocopherol and from 0.3 to 3% polysorbate 80 e.g. 4.3% squalene, 4.7% α-tocopherol, 1.9% polysorbate 80. The weight ratio of squalene:tocopherol is preferably ≤1 (e.g. 0.90) as this provides a more stable emulsion. Squalene and polysorbate 80 may be present volume ratio of about 5:2, or at a weight ratio of about 11:5. One such emulsion can be made by dissolving polysorbate 80 in PBS to give a 2% solution, then mixing 90 ml of this solution with a mixture of (5 g of DL-α-tocopherol and 5 ml squalene), then microfluidizing the mixture. The resulting emulsion may have submicron oil droplets e.g. with a size between 100 and 250 nm, preferably about 180 nm.
An emulsion of squalene, a tocopherol, and a Triton detergent (e.g. Triton X-100). The emulsion may also include a 3-O-deacylated monophosphoryl lipid A (‘3d-MPL’). The emulsion may contain a phosphate buffer.
   An emulsion comprising squalene, a polysorbate (e.g. polysorbate 80), a Triton detergent (e.g. Triton X-100) and a tocopherol (e.g. an α-tocopherol succinate). The emulsion may include these three components at a mass ratio of about 75:11:10 (e.g. 750 μg/ml polysorbate 80, 1 10 μg/ml Triton X-100 and 100 μg/ml α-tocopherol succinate), and these concentrations should include any contribution of these components from antigens. The emulsion may also include a 3d-MPL. The emulsion may also include a saponin, such as QS21. The aqueous phase may contain a phosphate buffer.
An emulsion comprising squalene, an aqueous solvent, a polyoxyethylene alkyl ether hydrophilic nonionic surfactant (e.g. polyoxyethylene cetostearyl ether) and a hydrophobic nonionic surfactant (e.g. a sorbitan ester or mannide ester, such as sorbitan monoleate or ‘Span 80’). The emulsion is preferably thermoreversible and/or has at least 90% of the oil droplets (by volume) with a size less than 200 nm (US-2007/0014805). The emulsion may also include one or more of: alditol; a cryoprotective agent (e.g. a sugar, such as dodecylmaltoside and/or sucrose); and/or an alkylpolyglycoside. It may also include a TLR4 agonist, such as one whose chemical structure does not include a sugar ring (WO2007/080308). Such emulsions may be lyophilized.

The compositions of these emulsions, expressed above in percentage terms, may be modified by dilution or concentration (e.g. by an integer, such as 2 or 3 or by a fraction, such as ⅔ or ¾), in which their ratios stay the same. For instance, a 2-fold concentrated MF59® would have about 10% squalene, about 1% polysorbate 80 and about 1% sorbitan trioleate. Concentrated forms can be diluted (e.g. with an antigen solution) to give a desired final concentration of emulsion.

Emulsions of the invention are ideally stored at between 2° C. and 8° C. They should not be frozen. They should ideally be kept out of direct light. In particular, squalene-containing emulsions and vaccines of the invention should be protected to avoid photochemical breakdown of squalene. If emulsions of the invention are stored then this is preferably in an inert atmosphere e.g. N2 or argon.

### Vaccines

Although it is possible to administer oil-in-water emulsion adjuvants on their own to patients (e.g. to provide an adjuvant effect for an antigen that has been separately administered to the patient), it is more usual to admix the adjuvant with an antigen prior to administration, to form an immunogenic composition e.g. a vaccine. Mixing of emulsion and antigen may take place extemporaneously, at the time of use, or can take place during vaccine manufacture, prior to filling. The methods of the invention can be applied in both situations.

Thus, a method of the invention may include a further process step of admixing the emulsion with an antigen component. As an alternative, it may include a further step of packaging the adjuvant into a kit as a kit component together with an antigen component.

Overall, therefore, the invention can be used when preparing mixed vaccines or when preparing kits including antigen and adjuvant ready for mixing. Where mixing takes place during manufacture then the volumes of bulk antigen and emulsion that are mixed will typically be greater than 1 liter e.g. ≥5 liters, ≥10 liters, ≥20 liters, ≥50 liters, ≥100 liters, ≥250 liters, etc. Where mixing takes place at the point of use then the volumes that are mixed will typically be smaller than 1 milliliter e.g. ≤0.6 ml, ≤0.5 ml, ≤0.4 ml, ≤0.3 ml, ≤0.2 ml, etc. In both cases it is usual for substantially equal volumes of emulsion and antigen solution to be mixed i.e. substantially 1:1 (e.g. between 1.1:1 and 1:1.1, preferably between 1.05:1 and 1:1.05, and more preferably between 1.025:1 and 1:1.025). In some embodiments, however, an excess of emulsion or an excess of antigen may be used (WO2007/052155). Where an excess volume of one component is used, the excess will generally be at least 1.5:1 e.g. ≥2:1, ≥2.5:1, ≥3:1, ≥4:1, ≥5:1, etc.

Where antigen and adjuvant are presented as separate components within a kit, they are physically separate from each other within the kit, and this separation can be achieved in various ways. For instance, the components may be in separate containers, such as vials. The contents of two vials can then be mixed when needed e.g. by removing the contents of one vial and adding them to the other vial, or by separately removing the contents of both vials and mixing them in a third container.

In another arrangement, one of the kit components is in a syringe and the other is in a container such as a vial. The syringe can be used (e.g. with a needle) to insert its contents into the vial for mixing, and the mixture can then be withdrawn into the syringe. The mixed contents of the syringe can then be administered to a patient, typically through a new sterile needle. Packing one component in a syringe eliminates the need for using a separate syringe for patient administration.

In another preferred arrangement, the two kit components are held together but separately in the same syringe e.g. a dual-chamber syringe (WO2005/089837; U.S. Pat. No. 6,692,468; WO00/07647; WO99/17820; U.S. Pat. Nos. 5,971,953; 4,060,082; EP-A-0520618; WO98/01174). When the syringe is actuated (e.g. during administration to a patient) then the contents of the two chambers are mixed. This arrangement avoids the need for a separate mixing step at time of use.

The contents of the various kit components will generally all be in liquid form. In some arrangements, a component (typically the antigen component rather than the emulsion component) is in dry form (e.g. in a lyophilized form), with the other component being in liquid form. The two components can be mixed in order to reactivate the dry component and give a liquid composition for administration to a patient. A lyophilized component will typically be located within a vial rather than a syringe. Dried components may include stabilizers such as lactose, sucrose or mannitol, as well as mixtures thereof e.g. lactose/sucrose mixtures, sucrose/mannitol mixtures, etc. One possible arrangement uses a liquid emulsion component in a pre-filled syringe and a lyophilized antigen component in a vial.

If vaccines contain components in addition to emulsion and antigen then these further components may be included in one or two kit components, or may be part of a third kit component.

Suitable containers for mixed vaccines of the invention, or for individual kit components, include vials and disposable syringes. These containers should be sterile.

Where a composition/component is located in a vial, the vial is preferably made of a glass or plastic material. The vial is preferably sterilized before the composition is added to it. To avoid problems with latex-sensitive patients, vials are preferably sealed with a latex-free stopper, and the absence of latex in all packaging material is preferred. In one embodiment, a vial has a butyl rubber stopper. The vial may include a single dose of vaccine/component, or it may include more than one dose (a ‘multidose’ vial) e.g. 10 doses. In one embodiment, a vial includes 10×0.25 ml doses of emulsion. Preferred vials are made of colorless glass.

A vial can have a cap (e.g. a Luer lock) adapted such that a pre-filled syringe can be inserted into the cap, the contents of the syringe can be expelled into the vial (e.g. to reconstitute lyophilized material therein), and the contents of the vial can be removed back into the syringe. After removal of the syringe from the vial, a needle can then be attached and the composition can be administered to a patient. The cap is preferably located inside a seal or cover, such that the seal or cover has to be removed before the cap can be accessed.

Where a composition/component is packaged into a syringe, the syringe will not normally have a needle attached to it, although a separate needle may be supplied with the syringe for assembly and use. Safety needles are preferred. 1-inch 23-gauge, 1-inch 25-gauge and ⅝-inch 25-gauge needles are typical. Syringes may be provided with peel-off labels on which the lot number, influenza season and expiration date of the contents may be printed, to facilitate record keeping. The plunger in the syringe preferably has a stopper to prevent the plunger from being accidentally removed during aspiration. The syringes may have a latex rubber cap and/or plunger. Disposable syringes contain a single dose of vaccine. The syringe will generally have a tip cap to seal the tip prior to attachment of a needle, and the tip cap is preferably made of a butyl rubber. If the syringe and needle are packaged separately then the needle is preferably fitted with a butyl rubber shield.

The emulsion may be diluted with a buffer prior to packaging into a vial or a syringe. Typical buffers include: a phosphate buffer; a Tris buffer; a borate buffer; a succinate buffer; a histidine buffer; or a citrate buffer. Dilution can reduce the concentration of the adjuvant's components while retaining their relative proportions e.g. to provide a “half-strength” adjuvant.

Containers may be marked to show a half-dose volume e.g. to facilitate delivery to children. For instance, a syringe containing a 0.5 ml dose may have a mark showing a 0.25 ml volume.

Where a glass container (e.g. a syringe or a vial) is used, then it is preferred to use a container made from a borosilicate glass rather than from a soda lime glass.

Various antigens can be used with oil-in-water emulsions, including but not limited to: viral antigens, such as viral surface proteins; bacterial antigens, such as protein and/or saccharide antigens; fungal antigens; parasite antigens; and tumor antigens. The invention is particularly useful for vaccines against influenza virus, HIV, hookworm, hepatitis B virus, herpes simplex virus, rabies, respiratory syncytial virus, cytomegalovirus, Staphylococcus aureus, chlamydia, SARS coronavirus, varicella zoster virus, Streptococcus pneumoniae, Neisseria meningitidis, Mycobacterium tuberculosis, Bacillus anthracis, Epstein Barr virus, human papillomavirus, etc. For example:
Influenza virus antigens. These may take the form of a live virus or an inactivated virus. Where an inactivated virus is used, the vaccine may comprise whole virion, split virion, or purified surface antigens (including hemagglutinin and, usually, also including neuraminidase). Influenza antigens can also be presented in the form of virosomes. The antigens may have any hemagglutinin subtype, selected from H1, H2, H3, H4, H5, H6, H7, H8, H9, H10, H11, H12, H13, H14, H15 and/or H16. Vaccine may include antigen(s) from one or more (e.g. 1, 2, 3, 4 or more) influenza virus strains, including influenza A virus and/or influenza B virus, e.g. a monovalent A/H5N1 or A/H1N1 vaccine, or a trivalent A/H1N1+A/H3N2+B vaccine. The influenza virus may be a reassortant strain, and may have been obtained by reverse genetics techniques (Hoffmann et al. (2002) Vaccine 20:3165-3170; Subbarao et al. (2003) Virology 305:192-200; Liu et al. (2003) Virology 314:580-590; Ozaki et al. (2004) J. Virol. 78:1851-1857; Webby et al. (2004) Lancet 363:1099-1103). Thus, the virus may include one or more RNA segments from a A/PR/8/34 virus (typically 6 segments from A/PR/8/34, with the HA and N segments being from a vaccine strain, i.e. a 6:2 reassortant). The viruses used as the source of the antigens can be grown either on eggs (e.g. embryonated hen eggs) or on cell culture. Where cell culture is used, the cell substrate will typically be a mammalian cell line, such as MDCK; CHO; 293T; BHK; Vero; MRC-5; PER.C6; WI-38; etc. Preferred mammalian cell lines for growing influenza viruses include: MDCK cells (WO97/37000; Brands et al. (1999) Dev Biol Stand 98:93-100; Halperin et al. (2002) Vaccine 20:1240-7; Tree et al. (2001) Vaccine 19:3444-50), derived from Madin Darby canine kidney; Vero cells (Istner et al. (1998) Vaccine 16:960-8; Kistner et al. (1999) Dev Biol Stand 98:101-110; Bruhl et al. (2000) Vaccine 19:1149-58), derived from African green monkey kidney; or PER.C6 cells (Pau et al. (2001) Vaccine 19:2716-21), derived from human embryonic retinoblasts. Where virus has been grown on a mammalian cell line then the composition will advantageously be free from egg proteins (e.g. ovalbumin and ovomucoid) and from chicken DNA, thereby reducing allergenicity. Unit doses of vaccine are typically standardized by reference to hemagglutinin (HA) content, typically measured by SRID. Existing vaccines typically contain about 15 μg of HA per strain, although lower doses can be used, particularly when using an adjuvant. Fractional doses such as ‘A (i.e. 7.5 μg HA per strain), ‘A and Vs have been used (WO01/22992; Hebe et al. (2004) Virus Res. 103(1-2):163-71), as have higher doses (e.g. 3× or 9× doses (Treanor et al. (1996) J Infect Dis 173:1467-70; Keitel et al. (1996) Clin Diagn Lab Immunol 3:507-10). Thus, vaccines may include between 0.1 and 150 μg of HA per influenza strain, preferably between 0.1 and 50 μg e.g. 0.1-20 μg, 0.1-15 μg, 0.1-10 μg, 0.1-7.5 μg, 0.5-5 μg, etc. Particular doses include e.g. about 15, about 10, about 7.5, about 5, about 3.8, about 3.75, about 1.9, about 1.5, etc. per strain.
Human immunodeficiency virus, including HIV-1 and HIV-2. The antigen will typically be an envelope antigen.
Hepatitis B virus surface antigens. This antigen is preferably obtained by recombinant DNA methods e.g. after expression in a Saccharomyces cerevisiae yeast. Unlike native viral HBsAg, the recombinant yeast-expressed antigen is non-glycosylated. It can be in the form of substantially-spherical particles (average diameter of about 20 nm), including a lipid matrix comprising phospholipids. Unlike native HBsAg particles, the yeast-expressed particles may include phosphatidylinositol. The HBsAg may be from any of subtypes aywl, ayw2, ayw3, ayw4, ayr, adw2, adw4, adrq− and adrq+.
Hookworm, particularly as seen in canines (Ancylostoma caninum). This antigen may be recombinant Ac-MTP-1 (astacin-like metalloprotease) and/or an aspartic hemoglobinase (Ac-APR-1), which may be expressed in a baculovirus/insect cell system as a secreted protein (Williamson et al. (2006) Infection and Immunity 74:961-7; Loukas et al. (2005) PLoS Med 2(10): e295),
Herpes simplex virus antigens (HSV). A preferred HSV antigen for use with the invention is membrane glycoprotein gD. It is preferred to use gD from a HSV-2 strain (‘gD2’ antigen). The composition can use a form of gD in which the C-terminal membrane anchor region has been deleted (EP-A-0139417) e.g. a truncated gD comprising amino acids 1-306 of the natural protein with the addition of asparagine and glutamine at the C-terminus. This form of the protein includes the signal peptide which is cleaved to yield a mature 283 amino acid protein. Deletion of the anchor allows the protein to be prepared in soluble form.
Human papillomavirus antigens (HPV). Preferred HPV antigens for use with the invention are LI capsid proteins, which can assemble to form structures known as virus-like particles (VLPs). The VLPs can be produced by recombinant expression of LI in yeast cells (e.g. in S. cerevisiae) or in insect cells (e.g. in Spodoptera cells, such as S. frugiperda, or in Drosophila cells). For yeast cells, plasmid vectors can carry the LI gene(s); for insect cells, baculovirus vectors can carry the LI gene(s). More preferably, the composition includes LI VLPs from both HPV-16 and HPV-18 strains. This bivalent combination has been shown to be highly effective (Harper et al. (2004) Lancet 364(9447):1757-65). In addition to HPV-16 and HPV-18 strains, it is also possible to include LI VLPs from HPV-6 and HPV-11 strains. The use of oncogenic HPV strains is also possible. A vaccine may include between 20-60 μg/ml (e.g. about 40 μg/ml) of LI per HPV strain.
Anthrax antigens. Anthrax is caused by Bacillus anthracis. Suitable B. anthracis antigens include A-components (lethal factor (LF) and edema factor (EF)), both of which can share a common B-component known as protective antigen (PA) (J Toxicol Clin Toxicol (2001) 39:85-100; Demicheli et al. (1998) Vaccine 16:880-884; Stepanov et al. (1996) J Biotechnol 4AΛ55A60). The antigens may optionally be detoxified (J Toxicol Clin Toxicol (2001) 39:85-100; Demicheli et al. (1998) Vaccine 16:880-884; Stepanov et al. (1996) J Biotechnol 4AΛ55A60).
S. aureus antigens. A variety of S. aureus antigens are known. Suitable antigens include capsular saccharides (e.g. from a type 5 and/or type 8 strain) and proteins (e.g. IsdB, Hla, etc.). Capsular saccharide antigens are ideally conjugated to a carrier protein.
S. pneumoniae antigens. A variety of S. pneumoniae antigens are known. Suitable antigens include capsular saccharides (e.g. from one or more of serotypes 1, 4, 5, 6B, 7F, 9V, 14, 18C, 19F, and/or 23F) and proteins (e.g. pneumolysin, detoxified pneumolysin, polyhistidine triad protein D (PhtD), etc.). Capsular saccharide antigens are ideally conjugated to a carrier protein.
Cancer antigens. A variety of tumour-specific antigens are known. The invention may be used with antigens that elicit an immunotherapeutic response against lung cancer, melanoma, breast cancer, prostate cancer, etc.

A solution of the antigen will normally be mixed with the emulsion e.g. at a 1:1 volume ratio. This mixing can either be performed by a vaccine manufacturer, prior to filling, or can be performed at the point of use, by a healthcare worker.

### Pharmaceutical Compositions

A solution of the antigen will normally be mixed with the emulsion e.g. at a 1:1 volume ratio. This mixing can either be performed by a vaccine manufacturer, prior to filling, or can be performed at the point of use, by a healthcare worker.

Compositions made using the methods of the invention are pharmaceutically acceptable. They may include components in addition to the emulsion and the optional antigen.

The composition may include a preservative such as thiomersal or 2-phenoxyethanol. It is preferred, however, that the vaccine should be substantially free from (i.e. less than 5 μg/ml) mercurial material e.g. thiomersal-free (Banzhoff (2000) Immunology Letters 71:91-96; WO02/097072). Vaccines and components containing no mercury are more preferred.

The pH of a composition will generally be between 5.0 and 8.1, and more typically between 6.0 and 8.0 e.g. between 6.5 and 7.5. A process of the invention may therefore include a step of adjusting the pH of the vaccine prior to packaging.

The composition is preferably sterile. The composition is preferably non-pyrogenic e.g. containing <1 EU (endotoxin unit, a standard measure) per dose, and preferably <0.1 EU per dose. The composition is preferably gluten free.

The composition may include material for a single immunization, or may include material for multiple immunizations (i.e. a ‘multidose’ kit). The inclusion of a preservative is preferred in multidose arrangements.

Vaccines are typically administered in a dosage volume of about 0.5 ml, although a half dose (i.e. about 0.25 ml) may be administered to children.

### Methods of Treatment, and Administration of the Vaccine

Vaccines are typically administered in a dosage volume of about 0.5 ml, although a half dose (i.e. about 0.25 ml) may be administered to children.

The invention provides kits and compositions prepared using the methods of the invention. The compositions prepared according to the methods of the invention are suitable for administration to human patients, and the invention provides a method of raising an immune response in a patient, comprising the step of administering such a composition to the patient.

The invention also provides these kits and compositions for use as medicaments.

The invention also provides the use of: (i) an aqueous preparation of an antigen; and (ii) an oil-in-water emulsion prepared according to the invention, in the manufacture of a medicament for raising an immune response in a patient.

The immune response raised by these methods and uses will generally include an antibody response, preferably a protective antibody response.

The compositions can be administered in various ways. The most preferred immunization route is by intramuscular injection (e.g. into the arm or leg), but other available routes include subcutaneous injection, intranasal (Greenbaum et al. (2004) Vaccine 22:2566-77; Zurbriggen et al. (2003) Expert Rev Vaccines 2:295-304; Piascik (2003) J Am Pharm Assoc (Wash D.C.). 43:728-30), oral (Mann et al. (2004) Vaccine 22:2425-9), intradermal (Halperin et al. (1979) Am J Public Health 69:1247-50; Herbert et al. (1979) J Infect Dis 140:234-8), transcutaneous, transdermal (Chen et al. (2003) Vaccine 21:2830-6), etc.

Vaccines prepared according to the invention may be used to treat both children and adults. The patient may be less than 1 year old, 1-5 years old, 5-15 years old, 15-55 years old, or at least 55 years old. The patient may be elderly (e.g. ≥50 years old, preferably ≥65 years), the young (e.g. <5 years old), hospitalized patients, healthcare workers, armed service and military personnel, pregnant women, the chronically ill, immunodeficient patients, and people travelling abroad. The vaccines are not suitable solely for these groups, however, and may be used more generally in a population.

Vaccines of the invention may be administered to patients at substantially the same time as (e.g. during the same medical consultation or visit to a healthcare professional) other vaccines.

### Intermediate Processes

The invention also provides a method for the manufacture of an oil-in-water emulsion, comprising microfluidization of a first emulsion to form a second emulsion and then filtration of the second emulsion. The first emulsion has the characteristics described above.

The invention also provides a method for the manufacture of an oil-in-water emulsion, comprising filtration of a second emulsion, i.e. a microfluidized emulsion. The microfluidised emulsion has the characteristics described above.

The invention also provides a method for the manufacture of a vaccine, comprising combining an emulsion with an antigen, where the emulsion has the characteristics described above.

### SPECIFIC EMBODIMENTS

Certain preferred embodiments of the present disclosure are summarized in the following paragraphs. This list is exemplary and not exhaustive of all of the embodiments provided by this disclosure.

Embodiment 1. A method of improving filtration of an oil-in water emulsion through a membrane filter comprising filtering the oil-in water emulsion through a membrane filter at a temperature less than or equal to 10° C., wherein the filter throughput is increased as compared to filtration of an oil-in water emulsion at a temperature of greater than 10° C. Embodiment 2. A method of preparing an oil-in-water emulsion, comprising filtering the oil-in-water emulsion through a membrane filter at a temperature less than or equal to 10° C., wherein the filter throughput is increased as compared to temperatures greater than 10° C.

Embodiment 3. A method of preparing an oil-in-water emulsion, comprising filtering the oil-in-water emulsion through a membrane filter at a filter throughput that is greater at temperatures below 10° C. than at temperatures above 10° C.

Embodiment 4. A method of preparing an oil-in-water emulsion adjuvant, comprising filtering the oil-in-water emulsion adjuvant through a membrane filter at an adjuvant throughput that is greater at a temperature of 5° C. than at a temperature above 10° C.

Embodiment 5. A method of preparing an oil-in-water emulsion, comprising mixing oil, an aqueous component, and a surfactant to form the oil-in-water emulsion; microfluidizing the mixture to reduce the average droplet size of the oil-in-water emulsion; and filtering the microfluidized oil-in-water emulsion through a membrane filter at a temperature of less than or equal to 10° C., wherein the filter throughput is increased as compared to filtration of an oil-in water emulsion at a temperature of greater than 10° C.

### General

The invention also provides a method for the manufacture of a vaccine, comprising combining an emulsion with an antigen, where the emulsion has the characteristics described above.

The term “comprising” encompasses “including” as well as “consisting” e.g. a composition “comprising” X may consist exclusively of X or may include something additional e.g. X+Y.

The word “substantially” does not exclude “completely” e.g. a composition which is “substantially free” from Y may be completely free from Y. Where necessary, the word “substantially” may be omitted from the definition of the invention.

The term “about” in relation to a numerical value x is optional and means, for example, x+10%.

Unless specifically stated, a process comprising a step of mixing two or more components does not require any specific order of mixing. Thus, components can be mixed in any order. Where there are three components then two components can be combined with each other, and then the combination may be combined with the third component, etc.

Where animal (and particularly bovine) materials are used in the culture of cells, they should be obtained from sources that are free from transmissible spongiform encephalopathies (TSEs), and in particular free from bovine spongiform encephalopathy (BSE). Overall, it is preferred to culture cells in the total absence of animal-derived materials.

All of the claims in the claim listing are herein incorporated by reference into the specification in their entireties as additional embodiments.

### EXAMPLES

### Example 1: Filter Determination for MF59® Filtration

Several filters including Express SHC, Express SHF, Durapore 0.22 μm, and Durapore 0.45/0.22 μm were tested during MF59® filtration to determine the optimum filter for enhancing filter capacity during both sizing and sterile filtration of the oil-in-water emulsion adjuvant. A description of the filters tested is shown below in Table 1.

TABLE 1
Filters tested during the MF59 ® sizing filtration trial

| | Filter | Catalogue | Filter Area |
|---|---|---|---|
| Filter | Details | Number | (cm²) |
| Express SHC | PES Sterilizing | SHGEA25NB6 | 3.5 |
| | Grade Filter | | |
| | (0.5/0.2 um) | | |
| Express SHC | PES M-Pleat | R&D | Varies |
| High Area | Sterilizing | | per |
| Scaling Tool | Grade Filter | | device |
| | (0.5/0.2 um) | | |
| Express SHF | PES Sterilizing | SGEPA25NB6 | 3.5 |
| | Grade Filter | | |
| | (0.2 um) | | |
| Durapore | PVDF Sterilizing | SVGLA25NB6 | 3.5 |
| 0.22 um | Grade Filter | | |
| | (0.22 um) | | |
| Durapore | PVDF Sterilizing | SHGLA25NB6 | 3.5 |
| 0.45/0.22 um | Grade Filter | | |
| | (0.45/0.22 um) | | |
| Milligard PES | PES Bioburden | SMP2A25NB6 | 3.5 |
| 0.2 um | Reduction Filter | | |
| | (1.2/0.2 um) | | |

The filtration Vmax (L/m²) for MF59® was measured for the above filters at various temperatures: 5° C., 30° C., and 40° C. All filters were run decoupled and under 43 psi constant pressure. Low pressure was 22 psi, and high pressure was 50 psi.

Briefly, the protocol for Vmax filtration involved first installing a filter device to a pressure vessel with a stop-lock upstream of the filter. Next, feed was added to the pressure vessel such that 1000 L/m² could be filtered. The device was vented to adequately remove air, and the vessel was pressurized.

Once filtration was initiated, time and volume were recorded at regular intervals. The trial was ended after all material was exhausted or >75% flux decay was observed.

For the tests performed at 5° C., study material was kept in a cold room then taken out and immediately filtered at ambient temperature. For the tests performed at 30° C. and 40° C., study material was warmed in a water bath then taken out and immediately filtered at ambient temperature.

The results of the Vmax study are shown in the below Table 2.

TABLE 2
Vmax results during MF59 ^{®} filtration study

| | | Test | Trial | | | Initial |
|---|---|---|---|---|---|---|
| | | Pressure | Loading | Trial Flux | Vmax | Flux |
| Temperature | Filter | (psi) | (L/m²) | Decay (%) | (L/m²) | (LMH) |
| 5 C. | SHF_Lot1 | 42.5 | 28.3 | 99.4 | 35.4 | 15860.2 |
| | SHF_Lot2 | 42.5 | 22.9 | 98.0 | 24.2 | 4751.3 |
| | SHC | 42.5 | 13.5 | 97.0 | 27.8 | 1254.5 |
| | SHC High Area | 42.5 | 15.7 | 99.8 | 17.1 | 25105.6 |
| | Durapore 0.22 um | 42.5 | 7.5 | 99.9 | 7.7 | 8431.3 |
| | Durapore | 42.5 | 12.1 | 99.6 | 13.2 | 12043.5 |
| | 0.45/0.22 um | | | | | |
| 30 C. | SHF_Lot 1 | 42.9 | 25.7 | 99.6 | 26.4 | 9466.0 |
| | SHF_Lot 2 | 40.2 | 20.1 | 100.4 | 21.4 | 9185.0 |
| | SHC_Lot 1 | 42.9 | 14.8 | 98.4 | 14.4 | 6112.3 |
| | SHC_Lot 2 | 40.2 | 16.9 | 100.0 | 16.8 | 12149.1 |
| | SHC High Area | 42.9 | 15.8 | 98.8 | 16.2 | 2423.5 |
| 40 C. | SHF_Lot1 | 42.9 | 21.8 | 100.0 | 21.9 | 23853.6 |
| | SHF_Lot2 | 42.9 | 20.7 | 100.0 | 20.5 | −81381.5 |
| | SHC_Lot1 | 42.9 | 15.4 | 99.6 | 16.1 | 4143.1 |
| | SHC_Lot2 | 42.9 | 19.2 | 100.2 | 19.8 | 8695.1 |
| | SHC High Area | 42.9 | 13.7 | 99.1 | 14.0 | 2661.1 |
| | Durapore | 42.9 | 12.0 | 99.6 | 12.4 | 4052.8 |
| | 0.45/0.22 um | | | | | |
| | Milligard PES | 22 | 122.3 | 53.9 | 683.5 | 656.1 |
| | 0.2 um_Low | | | | | |
| | Pressure | | | | | |
| | SHC post MG | 43.3 | 25.9 | 99.6 | 27.2 | 5819.8 |
| | PES 0.2 um | | | | | |
| | SHC_Lot 2_High | 49.9 | 32.5 | 99.7 | 33.9 | 8119.6 |
| | Pressure | | | | | |

The results of the Vmax study indicate that sterilizing grade filters were able to filter MF59® up to ˜30 L/m² at a 43 psi pressure drop. The SHF, SHC and Durapore filters showed improvements at 5° C. compared to 30° C. and 40° C. SHF had the highest filterability followed by SHC. SHF had a slightly better performance at lower temperature. Lot-to-lot variability between SHF and SHC was fairly low. The data suggests that increasing pressure increases filterability. For example, SHC showed about 1.5× improvement in capacity when pressure increased from 42.9 psi to 49.9 psi.

### CONCLUSIONS

In general, filtration of MF59® exhibits high rates of sterilizing grade filter fouling and shear thinning properties. Express SHF exhibited the most favorable filter hydraulics for all sterilizing grade filters tested. Express SHC High Area provided the lowest installation for processing a 330 L batch of MF59®, as the high area device provides double the area per cartridge with similar performance.

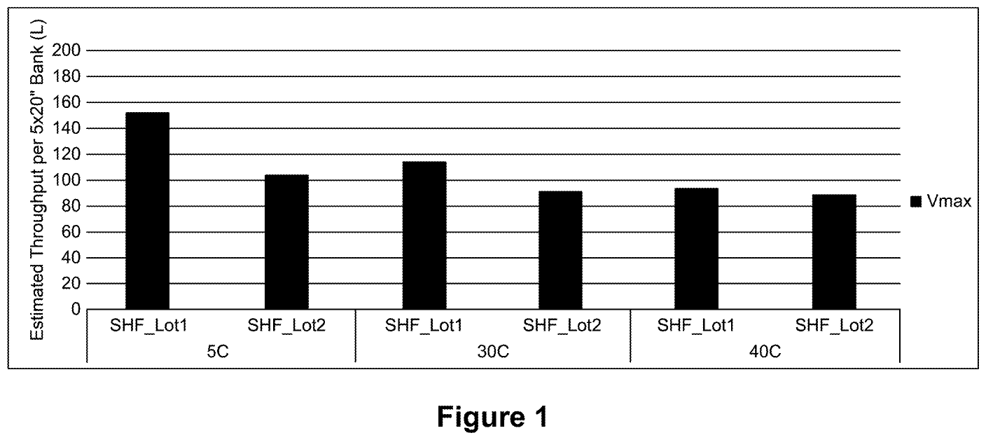

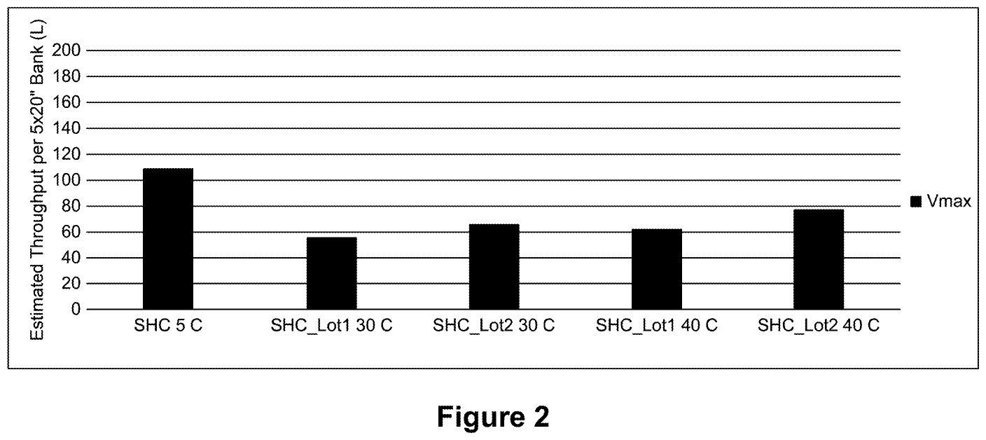

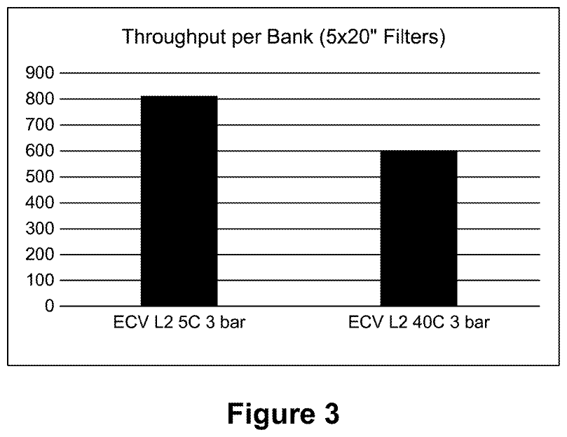

Separate filtration studies also demonstrated that throughput significantly increases when performed with various membranes at colder temperatures as shown in the below Tables 3 and 4 and in FIGS. 1-3.

TABLE 3
Additional Throughput Results Involving ECV

| | Pressure | Throughput |
|---|---|---|
| Filter | (Bar) | (L/m²) |
| ECV, 5° C. | 3 | 96.8 |
| ECV, 40° C. | 3 | 72.3 |
| ECV → ECV, 40° C. | 3 | 697.6 |
| ECV → ECV, 5° C. | 3 | >697.6 |

TABLE 4
Additional Throughput Results Involving Sartopore 2

| | MF59 ® | | |
|---|---|---|---|
| | Throughput (mL/cm²) at 90% flux decay | | |
| Temp | Sartopore 2 | Sartopore 2 XLG | Express SHC |
| 2-8° C. | 3.71 | 2.29 | 1.54 |
| 40° C. | 3.27 | 2.11 | 1.29 |

Table 5 shown below also demonstrated that increasing pressure further enhances throughput at colder temperature (e.g. 10° C.).

TABLE 5
Sartopore 2 Sterilizing Filter Throughput Summary

| | Throughput (L/m²) at V₈₀ | | | | | | | | |
|---|---|---|---|---|---|---|---|---|---|
| | 10° C. | | | 20° C. | | | 30° C. | | |
| MF59 ® Batch | 1 bar | 2 bar | 3 bar | 1 bar | 2 bar | 3 bar | 1 bar | 2 bar | 3 bar |
| 169069 | 79 | 420 | 845 | 43 | 170 | >366¹ | N/A | N/A | N/A |
| 167010 | 35 | 200 | 510 | N/A | N/A | N/A | 30 | 170 | 572 |

¹A logarithmic trendline could not accurately be established, so a linear trendline was used. The actual V₈₀ throughput is likely higher.

### REFERENCES

All references referred to are incorporated herein by reference in their entireties.

What is claimed is:

1. A method of improving filtration of an oil-in water emulsion through a membrane filter comprising filtering the oil-in water emulsion through a membrane filter at a temperature less than or equal to 10° C., wherein the filter throughput is increased as compared to filtration of an oil-in water emulsion at a temperature of greater than 10° C.

2. A method of preparing an oil-in-water emulsion, comprising filtering the oil-in-water emulsion through a membrane filter at a temperature less than or equal to 10° C., wherein the filter throughput is increased as compared to temperatures greater than 10° C.

3. The method according to any one of claims 1-2, wherein filtering the oil-in-water emulsion reduces the amount of bio-burden in oil-in-water emulsion.

4. The method according to any one of claims 1-2, wherein filtering the oil-in-water emulsion comprises sterile filtration of the oil-in-water emulsion.

5. The method according to any one of claims 1-2, wherein filtering the oil-in-water emulsion comprises particle size reduction filtration of the oil-in-water emulsion.

6. The method according to any one of claims 1-5 comprising filtering the oil-in-water emulsion through a membrane filter at a temperature from 2-8° C.

7. An oil-in-water emulsion prepared according to the method of any one of claims 1-6.

8. The oil-in-water emulsion according to claim 7, wherein the oil-in-water emulsion is an adjuvant.

9. The oil-in-water emulsion according to any one of claims 7-8, wherein the oil-in-water emulsion comprises squalene.

10. The oil-in-water emulsion according to any one of claims 7-9, wherein the oil-in-water emulsion comprises a submicron oil-in-water emulsion which includes (a) squalene, polysorbate 80 and sorbitan trioleate, or (b) squalene, a tocopherol and polysorbate 80.

11. The oil-in-water emulsion according to any one of claims 7-10, wherein the oil-in-water emulsion is MF59®.

12. A vaccine composition comprising an oil-in-water emulsion prepared according to the method of any one of claims 1-6.

13. The vaccine composition of claim 12, wherein the vaccine composition specifically targets influenza virus.

14. The vaccine composition according to any one of claims 12-13, wherein the oil-in-water emulsion is MF59®.

15. A method of preparing an oil-in-water emulsion adjuvant, comprising filtering the oil-in-water emulsion adjuvant through a membrane filter at an adjuvant throughput that is greater at a temperature of 5° C. than at a temperature above 10° C.

16. An oil-in-water emulsion adjuvant prepared according to the method of claim 15.

17. The oil-in-water emulsion adjuvant according to claim 16, wherein the oil-in-water emulsion adjuvant is MF59®.

18. A vaccine composition comprising an oil-in-water emulsion adjuvant prepared according to the method of claim 15.

19. The vaccine composition according to claim 18, wherein the vaccine composition specifically targets influenza virus.

20. The vaccine composition according to any one of claims 18-19, wherein the oil-in-water emulsion adjuvant is MF59®.

21. A method of preparing an oil-in-water emulsion, comprising
mixing oil, an aqueous component, and a surfactant to form the oil-in-water emulsion;
microfluidizing the mixture to reduce the average droplet size of the oil-in-water emulsion; and
filtering the microfluidized oil-in-water emulsion through a membrane filter at a temperature of less than or equal to 10° C., wherein the filter throughput is increased as compared to filtration of an oil-in water emulsion at a temperature of greater than 10° C.

22. The method according to claim 21, wherein the oil-in-water emulsion comprises squalene.

23. The method according to any one of claims 21-22, wherein mixing the oil, aqueous component, and surfactant comprises homogenizing the components.

24. An oil-in-water emulsion prepared according to the method of any one of claims 21-23.

25. The oil-in-water emulsion of claim 24, wherein the oil-in-water emulsion is MF59®.

26. A vaccine composition comprising an oil-in-water emulsion prepared according to the method of any one of claims 21-23.

27. The vaccine composition according to claim 26, wherein the vaccine composition specifically targets influenza virus.

28. The vaccine composition according to any one of claims 26-27, wherein the oil-in-water emulsion is MF59®.

29. The method according to any one of claims 1-23, further comprising admixing the oil-in-water emulsion with an antigen compound.

30. The method according to claim 29, wherein the antigen compound is an influenza virus antigen.

31. The method according to any one of claims 29-30, further comprising packaging the oil-in-water emulsion into a kit together with an antigen component.
